(19) United States
(12) Patent Application Publication
(10) Pub. No.: US 2007/0088078 A1
(43) Pub. Date: Apr. 19, 2007

(54) Methods for managing adipocyte fat accumulation

(76) Inventors: Slavik Dushenkov, Fort Lee, NJ (US); David Evans, Belle Mead, NJ (US); Kitty Ann Evans, Cocoa, FL (US)

Correspondence Address: JONES DAY, 222 EAST 41ST ST, NEW YORK, NY, 10017, US

(21) Appl. No.: 11/508,587

(22) Filed: Aug. 22, 2006

Related U.S. Application Data
(60) Provisional application No. 60/710,933, filed on Aug. 23, 2005.

(51) Int. Cl. A61K 31/353

(52) U.S. Cl. 514/456

(57) ABSTRACT

The present invention provides methods for management fat accumulation in an adipocyte using compositions comprising a polymethoxylated flavone fraction of at least one of 5,6,7,3′,4′-pentamethoxyflavone (sinensetin); 5,6,7,8,3′,4′-hexamethoxyflavone (nobiletin); 5,6,7,8,4′-pentamethoxyflavone (tangeretin); 5-hydroxy-6,7,8,3′,4′-pentamethoxyflavone (auranetin); 5-hydroxy-7,8,3′,4′-tetramethoxyflavone; 5,7-dihydroxy-6,8,3′,4′-tetramethoxyflavone; 5,7,8,3′,4′-pentamethoxyflavone; 5,7,8,4′-tetramethoxyflavone; 3,5,6,7,8,3′,4′-heptamethoxyflavone; 5-hydroxy-3,6,7,8,3′,4′-hexamethoxyflavone; 5-hydroxy-6,7,8,4′-tetramethoxyflavone; 5,6,7,4′-tetramethoxyflavone; 7-hydroxy-3,5,6,8,3′,4′-hexamethoxyflavone; or 7-hydroxy-3,5,6,3′,4′-pentamethoxyflavone. In certain embodiments, methods for preventing rebound weight gain in a subject who has experienced weight loss are provided.

### 1. RELATED APPLICATIONS

This application claims the benefit of U.S. Provisional Application No. 60/710,933, filed Aug. 23, 2005, which is incorporated herein by reference in its entirety.

### 2. TECHNICAL FIELD

The present invention relates to compositions and methods for managing fat accumulation and preventing weight gain. For example, the present invention relates to methods of using compositions comprising a polymethoxylated flavone to manage fat accumulation in adipocytes and to prevent adipogenesis. In some embodiments, methods for preventing rebound weight gain in a subject who has experienced weight loss are provided.

### 3. BACKGROUND

Obesity is an important unmet medical need and is increasing in prevalence. More than one-third of all Americans are clinically overweight and annual spending on weight control exceeds $30 billion.

In humans, obesity is characterized by an increase in the number (hyperplasia) as well as in the size (hypertrophy) of adipocytes, the cells in which fat is stored. New adipocytes arise through adipogenesis, a process by which specialized fibroblasts residing in adipose tissue (preadipocytes) are triggered to undergo adipocyte differentiation. De novo adipocyte differentiation can be initiated during the entire lifespan of mammals by recruiting fibroblastic precursors.

Anti-obesity drugs are a major objective of many pharmaceutical firms, and no effective drugs are currently on the market. Effective dietary supplements or drugs safe and useful for preventing or treating obesity are sought.

### 4. SUMMARY

In one aspect, the present invention provides methods for managing fat accumulation. In certain embodiments, the present invention provides methods for managing fat accumulation in an adipocyte. In some embodiments, the adipocyte is in a subject, preferably a mammal, more preferably a human. In particular, the methods provided comprise administering to a subject an effective amount of a composition comprising a polymethoxylated flavone (PMF) fraction as described below, thereby managing fat accumulation in an adipocyte in the subject.

In certain embodiments, methods are provided for managing fat accumulation in a human subject that is overweight or obese. In some embodiments, the human has had a history of unwanted weight gain, and is prone to becoming overweight or obese in absence of treatment with the methods for managing fat accumulation as described herein.

In certain embodiments, the methods provided prevent adipogenesis in a subject in need thereof.

In some embodiments, the methods provided prevent fat accumulation in a mature adipocyte in a subject in need thereof.

In certain embodiments, methods are provided for preventing fat accumulation in an adipocyte comprising contacting the adipocyte with a composition comprising a polymethoxylated flavone (PMF) fraction and a non-PMF fraction.

In some embodiments, the adipocyte is contacted with the composition in vitro.

In some embodiments, the adipocyte is contacted with the composition in a subject.

In another aspect, the present invention provides methods for preventing rebound weight gain in a subject in need thereof comprising administering to the subject an effective amount of a composition comprising a PMF fraction as described below, thereby preventing rebound weight gain.

A composition in accordance with the invention can comprise one PMF or a plurality of PMFs. In certain embodiments, the composition can comprise a PMF fraction and a non-PMF fraction, wherein the PMF fraction comprises one or more PMFs.

In certain embodiments, a composition of the invention comprises an orange peel extract. In some embodiments, the composition of the invention consists essentially of an orange peel extract. In general, the composition is not a natural source, such as, for instance, an orange peel.

In certain embodiments, the composition comprises about 30% (w/w) (dry weight) to about 75% (w/w) (dry weight) of a PMF fraction. In some embodiments, the composition comprises about 40% (w/w) (dry weight) to about 75% (w/w) (dry weight) of a PMF fraction. In some embodiments, the PMF fraction is in a range from about 0.5% (w/w) to about 5% (w/w), from about 1% (w/w) to about 10% (w/w), from about 10% (w/w) to about 20% (w/w), from about 20% (w/w) to about 30% (w/w), from about 30% (w/w) to about 40% (w/w), from about 40% (w/w) to about 50% (w/w), from about 50% (w/w) to about 60% (w/w), from about 60% (w/w) to about 70% (w/w), from about 70% (w/w) to about 80% (w/w), or from about 80% (w/w) to about 98% (w/w).

In some embodiments, the PMF fraction of a composition in accordance with the invention comprises at least one, at least two, at least three, at least four, at least five, at least six, at least seven, at least eight, at least nine, at least ten, at least eleven, at least twelve, at least thirteen or at least fourteen of the PMFs selected from the group consisting of 5,6,7,3′,4′-pentamethoxyflavone (sinensetin); 5,6,7,8,3′,4′-hexamethoxyflavone (nobiletin); 5,6,7,8,4′-pentamethoxyflavone (tangeretin); 5-hydroxy-6,7,8,3′,4′-pentamethoxyflavone (auranetin); 5-hydroxy-7,8,3′,4′-tetramethoxyflavone; 5,7-dihydroxy-6,8,3′,4′-tetramethoxyflavone; 5,7,8,3′,4′-pentamethoxyflavone; 5,7,8,4′-tetramethoxyflavone; 3,5,6,7,8,3′,4′-heptamethoxyflavone; 5-hydroxy-3,6,7,8,3′,4′-hexamethoxyflavone; 5-hydroxy-6,7,8,4′-tetramethoxyflavone; 5,6,7,4′-tetramethoxyflavone; 7-hydroxy-3,5,6,8,3′,4′-hexamethoxyflavone; and 7-hydroxy-3,5,6,3′,4′-pentamethoxyflavone.

In some embodiments, the PMF fraction of a composition in accordance with the invention consists of at least one, at least two, at least three, at least four, at least five, at least six, at least seven, at least eight, at least nine, at least ten, at least eleven, at least twelve, at least thirteen or at least fourteen of the PMFs selected from the group consisting of 5,6,7,3′,4′-pentamethoxyflavone (sinensetin); 5,6,7,8,3′,4′-hexamethoxyflavone (nobiletin); 5,6,7,8,4′-pentamethoxyflavone (tangeretin); 5-hydroxy-6,7,8,3′,4′-pentamethoxyflavone (auranetin); 5-hydroxy-7,8,3′,4′-tetramethoxyflavone; 5,7-dihydroxy-6,8,3′,4′-tetramethoxyflavone; 5,7,8,3′,4′-pentamethoxyflavone; 5,7,8,4′-tetramethoxyflavone; 3,5,6,7,8,3′,4′-heptamethoxyflavone; 5-hydroxy-3,6,7,8,3′,4′-hexamethoxyflavone; 5-hydroxy-6,7,8,4′-tetramethoxyflavone; 5,6,7,4′-tetramethoxyflavone; 7-hydroxy-3,5,6,8,3′,4′-hexamethoxyflavone; and 7-hydroxy-3,5,6,3′,4′-pentamethoxyflavone.

In some embodiments, a composition in accordance with the invention comprises a mixture of PMFs wherein the concentration of a PMF in composition is different from that in a natural source of the PMF or that the ratio of one PMF in the composition to that of another PMF in the composition is different from that in a natural source of the PMFs. Such a composition can be prepared, for example, by processing a natural source of PMFs, for instance an orange peel, such that at least one particular PMF has been selectively removed, retained or enriched. Alternatively, one or more isolated or synthesized PMF can be used to make such compositions or can be added to a processed form of a natural source of PMFs.

In some embodiments, a composition in accordance with the present invention comprises an orange peel extract.

In some embodiments, a composition in accordance with the invention can be a nutraceutical composition comprising one or more PMFs and a food, food additive, dietary supplement or medical food.

Typically, the amount of the composition administered to a subject in the methods of the invention can be from about 0.05 mg, about 0.1 mg, about 0.5 mg, about 1.0 mg, about 5 mg, about 10 mg, about 15 mg, about 20 mg, about 25 mg, about 30 mg, about 35 mg, about 40 mg, or about 50 mg, to about 75 mg, about 100 mg, about 200 mg, about 250 mg, about 300 mg, about 400 mg, about 500 mg, about 600 mg, about 700 mg, about 750 mg, about 800 mg, about 900 mg, about 1 g, about 2 g, about 3 g, or about 5 g per day.

In certain embodiments, the amount of PMF fraction in a composition administered to a subject in the methods provided herein ranges from about 0.05 mg, about 0.1 mg, about 0.5 mg, about 1.0 mg, about 5 mg, about 10 mg, about 15 mg, about 20 mg, about 25 mg, about 30 mg, about 35 mg, about 40 mg, or about 50 mg, to about 75 mg, about 100 mg, about 200 mg, about 250 mg, about 300 mg, about 400 mg, about 500 mg, about 600 mg, about 700 mg, about 750 mg, about 800 mg, about 900 mg, about 1 g, about 2 g, about 3 g, or about 5 g per day.

In some embodiments, the composition can be administered once, twice or more times per day. In certain embodiments, the composition can be administered on successive days for two, three or more days up to about seven days, about two weeks, about three weeks, about four weeks, about thirty days, about five weeks or about six weeks.

In some embodiments, the composition can be occasionally administered, for example, when the subject has, or intends to, ingest foods high in carbohydrate or fat content.

In some embodiments, the composition administered in the methods of the invention is administered via a buccal, nasal, oral, parenteral, rectal, sublingual, topical or transdermal route of administration.

In some embodiments, the composition administered in the methods of the invention is administered in an aerosol, chewable bar, bulk or loose dry form, capsule, cream, drink, elixir, emulsion, fluid, gel, granule, chewable gum, lotion, lozenge, ointment, paste, patch, pellet, powder, solution, spray, suppository, suspension, syrup, tablet, tea, tincture, vapor or wafer.

### 5. BRIEF DESCRIPTION OF THE DRAWINGS

FIG. 1 provides average absorbance (optical density units) observed in a colorimetric-based 3T3-L1 cell proliferation/cytotoxicity assay for cells treated with DMSO (control) or various concentrations of test composition as indicated. These results indicate that cell proliferation is inhibited in concentrations of approximately 20 μg/mL test composition or greater.

FIG. 2 provides observed levels of RNA expression of the indicated genes on days 0 (“growth”), 2, 4 and 10 in preadipocytes treated with differentiation factors in the absence of test composition.

FIG. 3 provides the percent changes in RNA expression levels of the indicated genes relative to the levels in control cells (see FIG. 2) on days 4 and 10 for cells treated with test composition.

FIG. 4 provides HMGA2 expression levels in cells treated with test composition.

FIG. 5 provides the weekly average of food consumed (in kcals) for mice on a standard diet, standard diet plus a PMF-containing composition, high fat diet, or a high fat diet plus a PMF-containing composition.

FIG. 6 provides the percent increase in body weight in mice fed a standard diet (SD), a standard diet supplemented with a PMF-containing composition (SD70% OPE), a high fat diet (HFD), and a high fat diet supplemented with a PMF-containing composition (HFD70% OPE).

FIG. 7 provides the percent body fat found in mice fed a standard diet (SD), a standard diet supplemented with a PMF-containing composition (SD70% OPE), a high fat diet (HFD), and a high fat diet supplemented with a PMF-containing composition (HFD70% OPE).

FIG. 8 provides the weights of individual fat pads from mice fed for sixteen weeks a standard diet (SD), a standard diet supplemented with a PMF-containing composition (SD70% OPE), a high fat diet (HFD), and a high fat diet supplemented with a PMF-containing composition (HFD70% OPE).

FIG. 9 provides results on the glycemic control of mice on various diets. Fasting glucoses levels (A) and glycated hemoglobin (HbA1c) (B) were determined in mice following a four hour fast.

FIG. 10 provides percent weight gain in mice by switching a high fat diet to a high fat diet plus 70% OPE and vice versa.

FIG. 11 provides the percentage of fat mass in mice on the indicated diets, including switched diets.

FIG. 12 provides the weights of individual fat pads in mice on the indicated diets, including switched diets.

### 6. TERMINOLOGY

The term “about” as used herein refers to a value that is no more than 10% above or below the value being modified by the term. For example, the term “about 5 minutes” means a range of from 4.5 minutes to 5.5 minutes.

As used herein, the term “composition” is meant to encompass pharmaceutical compositions, physiologically acceptable compositions and nutraceutical compositions. It will be understood that where a component, for example, a polymethoxylated flavone (PMF), in a “composition” also occurs in a natural source (for instance, orange peel), the term “composition” does not consist of the natural source (for instance, orange peel) of the component, but can, in certain embodiments, encompass a physically or chemically modified or processed form of the natural source, such as an extract of the natural source.

The term “effective amount” as used herein refers to the amount of a compound or composition that is sufficient to produce a desirable or beneficial effect when administered to a subject. In certain embodiments, an “effective amount” of a compound or composition prevents, delays, slows or reduces the accumulation of fat, e.g., triacylglycerides, in an adipocyte contacted with the compound or composition, or in an adipocyte in a subject that has been administered the compound or composition, as compared to an adipocyte not contacted the compound or composition, or adipocyte in a subject that has not been administered the compound or composition. In some embodiments, an “effective amount” of a compound or composition prevents, delays, slows or retards the progression of a preadipocyte contacted with the compound or composition into an adipocyte, or the progression of a preadipocyte into adipocyte in a subject that has been administered the compound or composition, as compared to a preadipocyte not contacted the compound or composition, or in a subject not administered with the compound or composition. In some embodiments, an “effective amount” of a compound or composition prevents, delays, slows or retards rebound weight gain in a subject that has been administered with the compound or composition as compared to a subject not administered with the compound or composition.

The term “isolated,” when used in context of a compound or composition that can be obtained from a natural source, refers to a compound or composition that is separated from one or more components from its natural source. Natural sources can be a fungus, plant or animal or a natural and unaltered product produced by a fungus, plant or animal including blood, cytosol, leaf, milk, mucous, peel, plasma, resin, rind, sap, sputum, stem, sweat, urine, and so forth. Thus, an “isolated” compound or composition is in a form such that its concentration or purity is greater than that in its natural source. For example, in certain embodiments, an “isolated” compound or composition can be obtained by purifying or partially purifying the compound or composition from a natural source. In some embodiments, an “isolated” compound or composition is obtained in vitro in a synthetic, biosynthetic or semisynthetic organic chemical reaction mixture.

As used herein, the terms “manage,” “managing” and “management” refer to the beneficial effects that a subject derives when the methods provided herein are practiced on the subject. In certain embodiments, a subject is administered a composition as described herein to “manage” fat accumulation so as to prevent, delay, retard, or reduce the accumulation of adipocyte fat in the subject in comparison to the subject not administered with the composition. In some embodiments, a subject is administered a composition as described herein to “manage” fat accumulation so as to prevent, delay, retard or reduce adipogenesis in the subject as compared to the subject not administered with the composition.

The term “polymethoxylated flavone” or “PMF” means, unless otherwise indicated, a compound having the formula 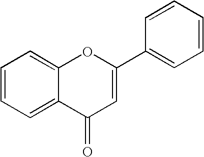
wherein at least one carbon, preferably two or more carbons, in the formula are attached to a —OCH₃ group (in place of one or more hydrogen atoms, not depicted in the formula) as valency permits. Optionally, substituents, such as, for example, hydroxyl, halide, monosaccharide, or other group, may be substituted onto one or more carbons not substituted with a methoxy group. For example, a “hydroxylated PMF” is a PMF that comprises one or more hydroxyl groups attached to a carbon not substituted with a methoxy group.

“Rebound weight gain” is the increase of body fat in a subject that occurs subsequent to the cessation or reduction by the subject of an exercise regimen, restricted calorie diet or drug (including tobacco) administration over a period in which the subject lost weight or maintained her or his weight. Typically, “rebound weight gain” can occur over a period of days, weeks, more typically, months up to a year, after the cessation or reduction of exercise, and/or of a restricted calorie diet or drug (including tobacco) administration by the subject.

“Solvate” refers to a compound, e.g., a PMF, that further includes a stoichiometric or non-stoichiometric amount of a solvent bound by non-covalent intermolecular forces. Where the solvent is water, the solvate is a hydrate.

As used herein, the term “subject” can refer to a mammal (e.g., a mouse, rat, guinea pig, rabbit, cow, pig, horse, donkey, goat, sheep, camel, cat, dog), more preferably a primate (e.g., a monkey, ape, gorilla, chimpanzee), and most preferably a human.

### 7. DETAILED DESCRIPTION

A primary function of adipocytes is the storage of triacylglycerides or fat during times when excess energy is available. As supported by the results described in the Examples section below, a composition comprising, e.g., enriched for, a polymethoxylated flavone (PMF) fraction can be effective for managing fat accumulation in a subject. In particular, without intending to be limited to any particular theory or mechanism, the results in the Examples section indicate that a PMF-containing composition can delay or prevent adipogenesis, as well as reduce or prevent fat accumulation into adipocytes. Methods for using a composition comprising a PMF fraction are described in Section 7.1. PMF containing compositions and methods for their preparation are described in Section 7.2.

7.1. Methods for Using PMF-Containing Compositions

In one aspect, the present invention provides methods of managing fat accumulation. In certain embodiments, the present invention provides for managing fat accumulation in an adipocyte. In some embodiments, the adipocyte is in a subject, preferably a mammal, more preferably a human. In some embodiments, the methods provided comprise administering to a subject an effective amount of a composition comprising a polymethoxylated flavone (PMF) fraction thereby managing fat accumulation in an adipocyte in the subject.

In certain embodiments, methods are provided for managing fat accumulation in a human subject that is overweight or obese. In some embodiments, the human has had a history of unwanted weight gain, and is prone to becoming overweight or obese in absence of treatment with the methods for managing fat accumulation as described herein.

In certain embodiments, the present invention provides methods of managing fat accumulation in a subject in need thereof wherein adipogenesis is managed, prevented, delayed, retarded or reduced.

In some embodiments, the present invention provides methods of managing fat accumulation in a subject in need thereof wherein fat accumulation in a mature adipocyte is managed, prevented, delayed, retarded or reduced.

In certain embodiments, the present methods of managing fat accumulation are for subjects in need of losing weight or in need of maintaining their body weight. Human subjects in need of losing weight typically have body mass index (BMI) above normal (i.e., ≧25). In some embodiments, a human subject has a BMI≧30, i.e., an obese subject.

In one aspect, the present invention provides methods of managing rebound weight gain in a subject in need thereof comprising administering to the subject an effective amount of a composition comprising a PMF fraction thereby managing rebound weight gain in the subject.

In certain embodiments, the present invention provides methods of managing rebound weight gain in a subject subsequent to the cessation or reduction by the subject of an exercise regimen, of a restricted calorie diet, or of drug (including tobacco) administration.

In one aspect, the present invention provides methods of losing weight, managing weight or preventing weight gain in a subject comprising administering to the subject an effective amount of a composition comprising a polymethoxylated flavone (PMF) fraction of at least about 40% (w/w) (dry weight) to about 75% (w/w) (dry weight). In certain embodiments, the composition comprises at least about 30% (w/w) (dry weight) to about 75% (w/w) (dry weight) of a PMF fraction.

In one aspect, methods are provided for increasing lean body or muscle mass in a subject comprising administering to the subject an amount of a composition effective to reduce fat accumulation in the subject wherein the composition comprises a polymethoxylated flavone (PMF) fraction of at least about 40% (w/w) (dry weight) to about 75% (w/w) (dry weight), thereby increasing lean body or muscle mass in the subject. In certain embodiments, the composition comprises at least about 30% (w/w) (dry weight) to about 75% (w/w) (dry weight) of a PMF fraction.

In another aspect, methods are provided for reducing waist circumference in a subject comprising administering to the subject an effective amount of a composition comprising a polymethoxylated flavone (PMF) fraction of at least about 40% (w/w) (dry weight) to about 75% (w/w) (dry weight). In certain embodiments, the composition comprises at least about 30% (w/w) (dry weight) to about 75% (w/w) (dry weight) of a PMF fraction

In one aspect, the present invention provides methods of increasing metabolic rate in a subject comprising administering to the subject an effective amount of a composition comprising a polymethoxylated flavone (PMF) fraction of at least about 40% (w/w) (dry weight) to about 75% (w/w) (dry weight). In certain embodiments, the composition comprises at least about 30% (w/w) (dry weight) to about 75% (w/w) (dry weight) of a PMF fraction.

In another aspect, the present invention provides methods of preventing fat accumulation in an adipocyte comprising contacting the adipocyte with a composition comprising a PMF fraction.

7.2. PMF-Containing Compositions and Methods for their Preparation

Compositions in accordance with the invention typically comprise a PMF fraction and a non-PMF fraction. In certain embodiments, the PMF fraction comprises one or more PMFs.

In certain embodiments, PMFs can be isolated, e.g., extracted, from a natural source for inclusion in compositions for use in the methods of the invention. In some embodiments a composition is an extract from a natural source comprising a PMF fraction. In some embodiments, compositions for use in the methods of the invention comprise an extract from cold-pressed orange peel oil solids. Preferably, compositions for use in the instant methods comprises an extract from Valencia and Hamlin varieties of oranges.

In some embodiments, a composition for use in the methods of the invention comprises a mixture of PMFs wherein the concentration of a PMF in the composition is different from that in a natural source of the PMF.

In some embodiments, the composition for use in the methods of the invention comprises a PMF fraction wherein a ratio of one PMF in the fraction to that of another PMF in the fraction is different from that in a natural source of the PMFs.

In certain embodiments, PMFs can be obtained synthetically for inclusion into compositions for use in methods of the invention. PMFs can be synthesized using any synthetic or semisynthetic technique, without limitation. A general synthetic scheme for flavones can found, for example, in Cushman and Nagarathnam (1990) Tetrahedron Letters 31: 6497-6500.

Generally, high Performance Liquid Chromatography (HPLC) can be useful either as a preparative technique for purifying or partially purifying PMFs or as an analytical technique for identifying PMFs in mixtures derived either from natural sources or synthetic reaction mixtures. For example, a Silica gel HPLC column (MacMod Analytical Co., Chadds Ford, Pa.) of dimensions 4.6 mm i.d.,×25 cm length was utilized using a solvent gradient from 10% to 90% chloroform (in hexane) (20 minutes) plus an additional 20 minutes run time holding at 90% chloroform to separate PMFs in an orange peel extract. Identification of PMFs by molecular weight was performed using Atmospheric Pressure Chemical Ionization mass spectrometry.

In certain embodiments, a composition for use in the methods of the invention comprises about 30% (w/w) (dry weight) to about 75% (w/w) (dry weight) of a PMF fraction. In certain embodiments, the composition comprises about 40% (w/w) (dry weight) to about 75% (w/w) (dry weight) of a PMF fraction. In some embodiments, the PMF fraction is in a range from about 0.5% (w/w) to about 5% (w/w), from about 1% (w/w) to about 110% (w/w), from about 110% (w/w) to about 20% (w/w), from about 20% (w/w) to about 30% (w/w), from about 30% (w/w) to about 40% (w/w), from about 40% (w/w) to about 50% (w/w), from about 50% (w/w) to about 60% (w/w), from about 60% (w/w) to about 70% (w/w), from about 70% (w/w) to about 80% (w/w), or from about 80% (w/w) to about 98% (w/w).

It will be understood that a PMF fraction can comprise one PMF or it can comprise more than one PMF.

In certain embodiments, a PMF fraction in a composition for use in the methods of the invention comprises at least one of 5,6,7,3′,4′-pentamethoxyflavone (sinensetin); 5,6,7,8,3′,4′-hexamethoxyflavone (nobiletin); 5,6,7,8,4′-pentamethoxyflavone (tangeretin); 5-hydroxy-6,7,8,3′,4′-pentamethoxyflavone (auranetin); 5-hydroxy-7,8,3′,4′-tetramethoxyflavone; 5,7-dihydroxy-6,8,3′,4′-tetramethoxyflavone; 5,7,8,3′,4′-pentamethoxyflavone; 5,7,8,4′-tetramethoxyflavone; 3,5,6,7,8,3′,4′-heptamethoxyflavone; 5-hydroxy-3,6,7,8,3′,4′-hexamethoxyflavone; 5-hydroxy-6,7,8,4′-tetramethoxyflavone; 5,6,7,4′-tetramethoxyflavone; 7-hydroxy-3,5,6,8,3′,4′-hexamethoxyflavone; or 7-hydroxy-3,5,6,3′,4′-pentamethoxyflavone.

In some embodiments, the PMF fraction of the composition for use in the methods of the invention comprises at least two, at least three, at least four, at least five, at least six, at least seven, at least eight, at least nine, at least ten, at least eleven, at least twelve, at least thirteen, or all of PMFs selected from the group consisting of 5,6,7,3′,4′-pentamethoxyflavone (sinensetin); 5,6,7,8,3′,4′-hexamethoxyflavone (nobiletin); 5,6,7,8,4′-pentamethoxyflavone (tangeretin); 5-hydroxy-6,7,8,3′,4′-pentamethoxyflavone (auranetin); 5-hydroxy-7,8,3′,4′-tetramethoxyflavone; 5,7-dihydroxy-6,8,3′,4′-tetramethoxyflavone; 5,7,8,3′,4′-pentamethoxyflavone; 5,7,8,4′-tetramethoxyflavone; 3,5,6,7,8,3′,4′-heptamethoxyflavone; 5-hydroxy-3,6,7,8,3′,4′-hexamethoxyflavone; 5-hydroxy-6,7,8,4′-tetramethoxyflavone; 5,6,7,4′-tetramethoxyflavone; 7-hydroxy-3,5,6,8,3′,4′-hexamethoxyflavone; and 7-hydroxy-3,5,6,3′,4′-pentamethoxyflavone.

In some embodiments, the composition for use in the methods of the invention consists of one or more PMFs.

In some embodiments, the composition for use in the methods of the invention consists essentially of one or more PMFs.

In some embodiments, the PMF fraction of the composition for use in the methods of the invention consists of one or more PMFs selected from the group consisting of 5,6,7,3′,4′-pentamethoxyflavone (sinensetin); 5,6,7,8,3′,4′-hexamethoxyflavone (nobiletin); 5,6,7,8,4′-pentamethoxyflavone (tangeretin); 5-hydroxy-6,7,8,3′,4′-pentamethoxyflavone (auranetin); 5-hydroxy-7,8,3′,4′-tetramethoxyflavone; 5,7-dihydroxy-6,8,3′,4′-tetramethoxyflavone; 5,7,8,3′,4′-pentamethoxyflavone; 5,7,8,4′-tetramethoxyflavone; 3,5,6,7,8,3′,4′-heptamethoxyflavone; 5-hydroxy-3,6,7,8,3′,4′-hexamethoxyflavone; 5-hydroxy-6,7,8,4′-tetramethoxyflavone; 5,6,7,4′-tetramethoxyflavone; 7-hydroxy-3,5,6,8,3′,4′-hexamethoxyflavone; and 7-hydroxy-3,5,6,3′,4′-pentamethoxyflavone.

In certain embodiments, the composition for use in the methods of the invention comprises a hydroxylated polymethoxylated flavone. In some embodiments, the composition comprises at least one of 5-hydroxy-6,7,8,3′,4′-pentamethoxyflavone (auranetin); 5-hydroxy-7,8,3′,4′-tetramethoxyflavone; 5,7-dihydroxy-6,8,3′,4′-tetramethoxyflavone; 5-hydroxy-3,6,7,8,3′,4′-hexamethoxyflavone; 5-hydroxy-6,7,8,4′-tetramethoxyflavone; 7-hydroxy-3,5,6,8,3′,4′-hexamethoxyflavone; or 7-hydroxy-3,5,6,3′,4′-pentamethoxyflavone.

In certain embodiments, the composition for use in the methods of the invention comprises a pentamethoxyflavone, a hexamethoxyflavone, a heptamethoxyflavone, or a octamethoxyflavone. In some embodiments, the composition comprises at least one of 5,6,7,8,3′,4′-hexamethoxyflavone (nobiletin); 3,5,6,7,8,3′,4′-heptamethoxyflavone; 5-hydroxy-3,6,7,8,3′,4′-hexamethoxyflavone; 7-hydroxy-3,5,6,8,3′,4′-hexamethoxyflavone; or 7-hydroxy-3,5,6,3′,4′-pentamethoxyflavone.

In certain embodiments, a composition for use in the methods of the invention comprises one or more hydroxylated PMFs. In some embodiments, a composition for use in the methods of the invention comprises a PMF fraction and one or more non-PMF-containing fractions, wherein the PMF fraction consists essentially of one or more hydroxylated PMFs.

In some embodiments, a composition, or a PMF fraction of a composition, for use in the methods provided herein consists essentially of one hyroxylated PMF, or at least two, at least three, at least four, at least five, at least six, or more hydroxylated PMFs, selected from the group consisting of 3-hydroxy-5,6,7,4′-tetramethoxyflavone; 3-hydroxy-5,6,7,8,4′-pentamethoxyflavone; 3-hydroxy-5,6,7,8,3′,4′-hexamethoxyflavone; 5-hydroxy-3,6,7,8,3′,4′-hexamethoxyflavone; 5-hydroxy-3,7,8,3′,4′-pentamethoxyflavone; 5-hydroxy-3,7,3′,4′-tetramethoxyflavone; 5-hydroxy-6,7,8,3′,4′-pentamethoxyflavone; 5-hydroxy-6,7,8,3′,4′,5′-hexamethoxyflavone; 5-hydroxy-6,7,8,4′-tetramethoxyflavone; 5-hydroxy-6,7,4′-trimethoxyflavone; 5,3′-dihydroxy-6,7,8,4′-tetramethoxyflavone; 5-hydroxy-7,8,3′,4′-tetramethoxyflavone; 5,7-dihydroxy-6,8,3′,4′-tetramethoxyflavone; 7-hydroxy-3,5,6,8,3′,4′-hexamethoxyflavone; 7-hydroxy-3,5,6,3′,4′-pentamethoxyflavone; 3′-hydroxy-5,6,7,4′-tetramethoxyflavone; 3′-hydroxy-5,6,7,8,4′-pentamethoxyflvone; 3′,4′-dihydroxy-5,6,7,8-tetramethoxyflavone; and 4′-hydroxy-5,6,7,8,3′-pentamethoxyflavone.

In certain embodiments, the composition for use in the methods of the invention does not contain detectable quantities of a liminoid. In some embodiments, the composition does not a detectable concentrations of limonin or nomalin.

7.2.1. Nutraceutical Formulations

In certain embodiments, a composition for use in the methods of the invention can be a nutraceutical composition. As used herein, the term “nutraceutical composition” refers to a composition comprising a food, food additive, dietary supplement, medical food or food for special dietary use and a PMF fraction.

In some embodiments, a nutraceutical composition of the invention typically comprises one or more consumable vehicles, carriers, excipients, or fillers. The term “consumable” means generally suitable for, or is approved by a regulatory agency of the Federal or a state government for, consumption by animals, and more particularly by humans.

As used herein, “food” means any substance, whether processed, semi-processed, or raw, which is intended for consumption by animals including humans, but does not include cosmetics, tobacco products or substances used only as pharmaceuticals.

As used herein, the term “dietary supplement” means a product (other than tobacco) intended to supplement the diet. Typically, a dietary supplement is a product that is labeled as a dietary supplement and is not represented for use as a conventional food or as a sole item of a meal or the diet. A dietary supplement can typically comprises one or more of the following dietary ingredients: a vitamin; a mineral; an herb or other botanical; an amino acid; a dietary supplement used by man to supplement the diet by increasing the total dietary intake; or a concentrate, metabolite, constituent, extract, or a combination of any of the ingredients. A dietary supplement can be consumed by a subject independent of any food, unlike a food additive which is incorporated into a food during the processing, manufacture, preparation, or delivery of the food, or just prior to its consumption.

As used herein, the term “medical food” refers to a food which is formulated to be consumed or administered enterally under the supervision of a physician and which is intended for the specific dietary management of a disease or condition for which distinctive nutritional requirements, based on recognized scientific principles, are established by medical evaluation. Examples of medical foods include but are not limited to sole source nutrition products which are complete nutritional products used to replace all other food intake; oral rehydration solutions for use in replacing fluids and electrolytes lost following diarrhea or vomiting; modular nutrient products containing specially selected components not intended to be complete nutritional sources but designed for the management of specific diseases and which have associated claims to effectiveness either direct or implied; and products intended for use in dietary management of inborn errors of metabolism.

As used herein, the term “food for special dietary use” refers to a food which purports or is represented to be used, for at least one of the following: supplying a special dietary need that exists by reason of a physical, physiological, pathological, or other condition, including but not limited to the condition of disease, convalescence, pregnancy, lactation, infancy, allergic hypersensitivity to food, underweight, overweight, or the need to control the intake of sodium; supplying a vitamin, mineral, or other ingredient for use by man to supplement his diet by increasing the total dietary intake; and supplying a special dietary need by reason of being a food for use as the sole item of the diet.

The nutraceutical compositions for use in the methods of the invention can also include one or more other ingredients that impart additional healthful or medicinal benefits.

In some embodiments, a nutraceutical composition for use in the methods of the invention comprises a PMF fraction and one or more “Generally Regarded As Safe” (“GRAS”) substance(s). Many GRAS substances are known and are listed in the various sections of the regulations of the United States public health authority, 21 CFR 73, 74, 75, 172, 173, 182, 184 and 186, which are incorporated herein by reference in their entirety.

In certain embodiments, the meaning of the term “medical food”, “food for special dietary use”, “dietary supplement” or “food additive” is the meaning of those terms as defined by a regulatory agency of a state government or the federal government of the United States, including the United States Food and Drug Administration.

In certain embodiments, the nutraceutical compositions for use in the methods of the invention comprise from about 0.001% to about 90%, by weight of a PMF fraction. Other amounts of the combination that are also contemplated are from about 0.0075% to about 75%, about 0.005% to about 50%, about 0.01% to about 35%, 0.1% to about 20%, 0.1% to about 15%, 1% to about 10%, and 2% to about 7%, by weight of the PMF fraction.

7.2.2. Pharmaceutical Formulations

In certain embodiments, pharmaceutical compositions comprising one or more PMFs and one or more physiologically acceptable carriers or excipients for use in accordance with the present invention may be formulated in conventional manner. Thus, the combination of one or more physiologically acceptable carriers or excipients and one or more PMFs and their physiologically acceptable salts and solvates may be formulated for administration by inhalation or insufflation (either through the mouth or the nose) oral, buccal, parenteral, rectal, or transdermal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the one or more PMFs.

For buccal administration the compositions may take the form of tablets or lozenges formulated in conventional manner.

For administration by inhalation, the pharmaceutical compositions for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the one or more PMFs and a suitable powder base such as lactose or starch.

The pharmaceutical composition may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The pharmaceutical composition may also be formulated in rectal compositions such as suppositories or retention enemas, e.g., containing conventional suppository bases such as cocoa butter or other glycerides.

The composition for use in the methods of invention can be formulated for transdermal or topical administration. For example, transdermal and topical dosage forms of the invention include, but are not limited to, ophthalmic solutions, sprays, aerosols, creams, lotions, ointments, gels, solutions, emulsions, suspensions, or other forms known to one of skill in the art. See, e.g., Remington: The Science and Practice of Pharmacy, 21^{st} ed., Lippincott, Williams & Wilkins, Philadelphia (2005); Ansel's Pharmaceutical Dosage Forms and Drug Delivery Systems, 8th ed., Lippincott, Williams & Wilkins, Philadelphia (2004). Transdermal dosage forms include “reservoir type” or “matrix type” patches, which can be applied to the skin and worn for a specific period of time to permit the penetration of a desired amount of active ingredients.

In addition to the formulations described previously, the pharmaceutical composition may also be formulated as a depot preparation. Such long acting formulations may be administered by implantation (for example subcutaneously or intramuscularly) or by intramuscular injection. Thus, for example, the complexes may be formulated with suitable polymeric or hydrophobic materials (for example as an emulsion in an acceptable oil) or ion exchange resins, or as sparingly soluble derivatives, for example, as a sparingly soluble salt.

The pharmaceutical composition may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

7.3. Administration of PMF-Containing Compositions

The amount of the composition to be administered to a subject in the methods of the invention, as well as the frequency of administration, will vary, for example, with the age, body weight, response and past medical history of the subject. Effective doses may be extrapolated from dose-response curves derived from in vitro or animal model test systems.

Generally, the active ingredient, i.e, the one or more PMFs, of the compositions used in methods of the invention are administered to a subject in amounts of about 0.001 μg/kg, about 0.005 μg/kg, about 0.01 μg/kg, about 0.05 μg/kg, about 0.1 μg/kg, about 0.5 μg/kg, about 1 μg/kg, about 5 μg/kg, about 10 μg/kg, about 50 μg/kg, about 100 μg/kg, about 500 μg/kg, about 1 mg/kg, about 5 mg/kg, about 10 mg/kg, about 25 mg/kg, about 50 mg/kg or about 75 mg/kg, where the units refer to micrograms (μg) of the active ingredient per subject body weight (kg).

In certain embodiments, where the composition is to be administered to a subject, preferably a human, in the methods of the invention, the amount of the PMF fraction in the administered composition is from about 0.05 mg, about 0.1 mg, about 0.5 mg, about 1.0 mg, about 5 mg, about 10 mg, about 15 mg, about 20 mg, about 25 mg, about 30 mg, about 35 mg, about 40 mg, or about 50 mg, to about 75 mg, about 100 mg, about 200 mg, about 250 mg, about 300 mg, about 400 mg, about 500 mg, about 600 mg, about 700 mg, about 750 mg, about 800 mg, about 900 mg, about 1 g, about 2 g, about 3 g, or about 5 g per day. In some embodiments, the amount of the PMF fraction in the administered composition is between about 50 mg to about 2 g per day.

In certain embodiments, the invention provides methods of managing fat accumulation in a subject with a dosing administration schedule sufficient to manage fat accumulation in a subject in need of such administration. In some embodiments, the composition can be administered once, twice or more times per day.

In certain embodiments, the composition can be administered on successive days for two, three or more days up to about seven days, about two weeks, about three weeks, about four weeks, about thirty days, about five weeks, about six weeks, about 7-12 weeks, or up to about one year or the continued life of the subject.

In certain embodiments, the composition can be administered to the subject once every two days, once every three days, once every four days, once every five days or once weekly, over a period of time of one week to about eight weeks.

In some embodiments, the composition can be occasionally administered, for example, when the subject has, or intends to, ingest foods high in carbohydrate or fat content.

The PMF containing composition can be administered by any suitable route that ensures bioavailability of the PMF fraction in the subject's circulation. Any route of administration that provides an effective amount of the PMF containing composition can be used. In particular, the route of administration can be indicated by the type of formulation, e.g., nutraceutical or pharmaceutical composition, as described above.

In some embodiments, the composition administered in the methods of the invention is administered via a buccal, nasal, oral, parenteral, rectal, sublingual, topical or transdermal route of administration.

7.4. Combination Methods

In another aspect, the present invention provides methods for managing fat accumulation in a subject in need thereof comprising administering to the subject a composition comprising one or more PMFs and a second agent (not a PMF-containing composition) effective for managing fat accumulation. In certain embodiments, the second agent promotes lean muscle mass development. Agents (not PMF-containing compositions) effective for managing fat accumulation or for promoting lean muscle mass development are known in the art and include, for example and without limitation, coleus forskohlii extract, ginko biloba leaf extract, glalangal rhizome extract, glucosamine, grape seed extract, green tea extract, manganese arginate, methionine, panththenic acid, vanadium, and so forth.

In certain embodiments, the second agent (not a PMF-containing composition) to be administered in combination methods of the invention is a gingerol, ECGC (epigallocatechin gallate), black tea extract, celery seed extract, garcinol, hawthorn fruit extract, hu zhang root extract, or citrin.

In some embodiments, the second agent (not a PMF-containing composition) to be administered in combination methods of the invention is a thermogenic agent (e.g., ECGC), a satiety promoter (e.g., fiber), an adipose absorption impediment (e.g., chitosan), a lean body mass promoter (e.g., conjugated linoleic acid or CLA), or a adipose prevention agent.

In certain embodiments, the PMF-containing composition and second agent (not a PMF-containing composition) are concurrently administered to the subject.

In some embodiments, the PMF-containing composition and second agent (not a PMF-containing composition) are sequentially administered to the subject.

In certain embodiments, the PMF-containing compositions are administered to prevent weight gain in subjects after having surgery to remove body fat. Surgery to remove body fat includes, for example, liposuction.

7.5. Biological Assays

Aspects of the compositions, including pharmaceutical and nutraceutical compositions, for use in the methods of the invention can routinely be tested in vitro, in a cell culture system, and/or in an animal model organism, such as a rodent animal model system, for a desired activity prior to use in humans. For example, assays can include cell culture assays in which a tissue sample is grown in culture, and exposed to or otherwise contacted with a PMF containing composition, and the effect of such composition upon the tissue sample is observed. Typically, preadipocytes, mature adipocytes or adipocyte-like cells such as 3T3-L1 cells can be used in such assays.

Compositions for use in the methods of the invention can be assayed for their ability to inhibit or induce the expression and/or activation of a gene product (e.g., cellular protein or RNA), as exemplified in the Examples section below. Since the temporal pattern of gene expression, at least for certain genes, that are required for adipogenesis are known, assaying compositions for their ability to inhibit or induce the expression of such genes is an appropriate indicator for assessing the compositions potential for preventing fat accumulation in an adipocyte. The inhibition or induction of the expression or activation of a gene product can be assayed by techniques known to those of skill in the art including, e.g., ELISAs flow cytometry, Northern blot analysis, Western blot analysis, RT-PCR kinase assays and electrophoretic mobility shift assays.

The toxicity and/or efficacy of the PMF-containing compositions for use in the methods of the invention can be determined by standard procedures in cell cultures or experimental animals, e.g., for determining the LD 50 (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. PMF-containing compositions that exhibit large therapeutic indices are preferred.

The data obtained from the cell culture assays and animal studies can be used in formulating a range of dosage of the PMF-containing compositions for use in humans. The dosage of such compositions lies preferably within a range of circulating concentrations that include the ED₅₀ with little or no toxicity. The dosage may vary within this range depending upon the dosage form employed and the route of administration utilized. For any composition used in the methods of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography (HPLC) and radioimmunasssay (RIA). The pharmacokinetics of a prophylactic or therapeutic composition can be determined, e.g., by measuring parameters such as peak plasma level (Cmax), area under the curve (AUC, which is measured by plotting plasma concentration of the composition versus time, and reflects bioavailability), half-life of the compound (t_{1/2}), and time at maximum concentration.

### 8. EXAMPLES

The following provides methods of preparing PMF-containing compositions and results of studies showing the anti-adipocyte neogenesis effect of such compositions.

The 3T3-L1 cell line used in the experiments described herein is an art recognized model for adipogenesis in humans. In vitro, confluent 3T3-L1 preadipocytes can be differentiated upon treatment with the combination of insulin, dexamethasone (DEX), and methylisobutylxanthine (MIX). See Student et al. (1980) J. Biol. Chem. 255:4745-4750. This adipogenic cocktail, containing MIX, DEX and insulin, is commonly abbreviated MDI. Gene induction is requisite for differentiation of preadipocytes into functional adipocytes.

8.1. Preparation of PMF-Containing Compositions

Orange solids, including peels, a byproduct from the orange juice industry, were obtained from Valencia and Hamlin varieties of oranges. Cold pressed orange peel oil was obtained by expressing oil from orange peel by pressing and clarifying the oil by centrifugation and winterizing (cooling) to precipitate waxes. Orange peel oil contains about 0.4% of a PMF fraction, a 98% light volatile fraction and 2% residue. The orange peel oil was concentrated approximately 30-fold by vacuum distillation, which was followed by ultra high vacuum distillation to achieve a volatiles recovery (VR) representing approximately a 50-fold increase in concentration. A separation process on the VR utilizing extraction with solvents followed by drying the extract was performed to yield an orange peel extract in powder form. The orange peel extract had a PMF fraction, representing approximately 70% (w/w) of the extract, and a non-PMF fraction. Typically, 1 kg cold pressed oil generates 3 g of 70% (w/w) orange peel extract. The non-PMF fraction was found to contain waxes, unsaturated fatty acids and β-sitosterol. No limonin was detected in the non-PMF fraction. The PMFs were analyzed by reverse phase high performance liquid chromatography (HPLC) and normal phase HPLC.

Typically, for normal phase HPLC, a silica gel HPLC column (MacMod Analytical Co., Chadds Ford, Pa.) with dimensions of 4.6 mm i.d.×25 cm length, was utilized with 90% hexane/10% chloroform starting solvent. Runs were performed using a 10% to 90% chloroform gradient over 20 minutes, followed by another 20 minutes at 90% chloroform. Mass spectrometry (MS) was used in conjunction with HPLC to identify individual PMFs. Atmospheric pressure chemical ionization MS was used for molecular weight determination. Standards were obtained from the Florida Department of Citrus (Lakeland, Fla.). The orange peel extract powder comprised a mixture of various analogs of methoxylated flavonoids, including:
5,6,7,3′,4′-pentamethoxyflavone (sinensetin);
5,6,7,8,3′,4′-hexamethoxyflavone (nobiletin);
5,6,7,8,4′-pentamethoxyflavone (tangeretin);
5-hydroxy-6,7,8,3′,4′-pentamethoxyflavone (auranetin);
5-hydroxy-7,8,3′,4′-tetramethoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; and
7-hydroxy-3,5,6,3′,4′-pentamethoxyflavone.

HPLC/MS was employed to identify polymethoxylated flavones in the orange peel extract. Standard PMFs were obtained from the Florida Department of Citrus (Lakeland, Fla.).

Prior to use in the following studies, extracts were diluted to an appropriate concentration in DMSO.

8.2. Cell Proliferation/Cytotoxicity Studies

A PMF-containing composition comprising a 70% PMF fraction (“70% OPE”) prepared as described in Section 7.1 above. The ability of the PMF-containing composition to inhibit cell proliferation and/or to have a cytotoxic effect was evaluated in a colorimetic assay that measures the number of viable cells. In this assay, decreased cell counts observed in treatment groups as compared to control groups can be a function of reduced cell proliferation or cytotoxicity.

Cell Preparation: Wells in a 96-well plate were seeded with 5,000 murine 3T3-L1 cells that were grown in the presence of orange peel extract (20 μg/mL) or DMSO (control) for 24 hours. Orange peel extract was tested in triplicate in two different plates. Wells were included that contained media and extract (no cells) for use in determining extract-specific, i.e., background, absorbance in the calorimetric assay.

Assay: A CELLTITER 96® AQUEOUS ONE SOLUTION cell proliferation assay (Promega, Madison Wis.) was performed as suggested by the manufacturer. In brief, calorimetric reagent was added directly to culture wells, incubated for 1-4 hours. Absorbance was recorded at 450 nm with a 96-well plate reader.

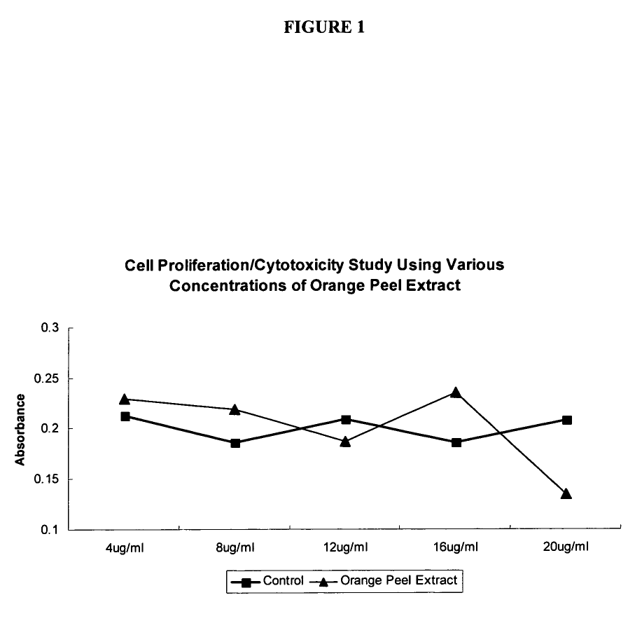

Results: The number of cells in cultures treated with 20 μg/mL orange peel extract was about 74% of the number of cells in control-treated cultures. Cell proliferation in cultures treated with concentrations of less then 20 μg/mL was similar to those treated with control (FIG. 1). Concentrations of orange peel extract of 100 μg/mL and greater appeared to be cytotoxic to cells.

8.3. Adipocyte Gene Expression/Cell Differentiation Assay

The effects on adipocyte differentiation of 70% OPE prepared as described above in Section 8.1 was assessed in cells by using real time PCT to monitor the relative levels of genetic markers known to accompany adipocyte differentiation.

Oil Red O Staining: Visual microscopic examination of the cells was performed after staining the cells with Oil Red O, which preferentially stains lipids in the cells. Briefly, cells were washed with phosphate-buffered saline (PBS) 2×, stained with 0.5% Oil Red O solution (60% isopropanol) for 15 min at room temperature, washed and removed with dye extraction buffer. The cells were examined under an optical microscope (Nikon Corporation, Tokyo, Japan), and the extent of staining was determined by visual scoring.

Treatment of 3T3-L1 Cells: Murine 3T3-L1 cells (American Type Culture Collection, Manassas, Va.) were grown in DMEM (pH 7.4) containing 10% FBS, 100 U/ml penicillin, and 100 g/ml streptomycin in a humidified 5% CO₂ atmosphere. For differentiation experiments, 200,000 cells were allowed to proliferate in growth medium for 2-3 days in 6-well plates, which were then subjected to standard differentiation-inducing agents (100 nM insulin, 1 μM dexamethasone and 250 μM MIX) in the presence of orange peel extract (20 μg/mL) or DMSO. Cells were collected on day 0 (“growth control”), and days 2, 4 and 10.

Gene Expression Detection: RNA was extracted from collected cells using the RNEASY MINI kit (Qiagen, Valencia Calif.) according to the manufacturer's instructions. cDNA was prepared from 1.5 micrograms of RNA using TAQMAN REVERSE TRANSCRIPTION kits (Applied Biosystems, Foster City Calif.). The cDNA was then used for real time PCR using SYBR® GREEN PCR core reagents (Applied Biosystems) and appropriate primers on a 7900HT sequence detection system (Applied Biosystems), according to the manufacturer's instructions, to determine the expression levels of adipocyte differentiation markers (“adipogenes”) peroxisome proliferator-activated receptor γ (PPARγ), CCAAT/enhancer binding protein α (CEBPα), adipoQ, ap2, HM-12, HM-21 and galectin-12. The data obtained were analyzed using the SDS2.1 software package (Applied Biosystems). In particular, analyses of the RNA expression were done using the comparative Ct (ΔΔCt) method with glyceraldehyde-3-phosphate dehydrogenase (GAPDH) used as a normalizer.

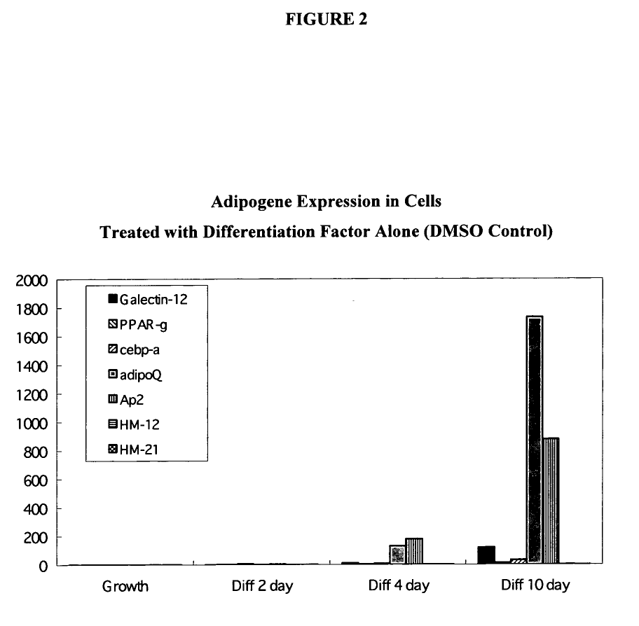

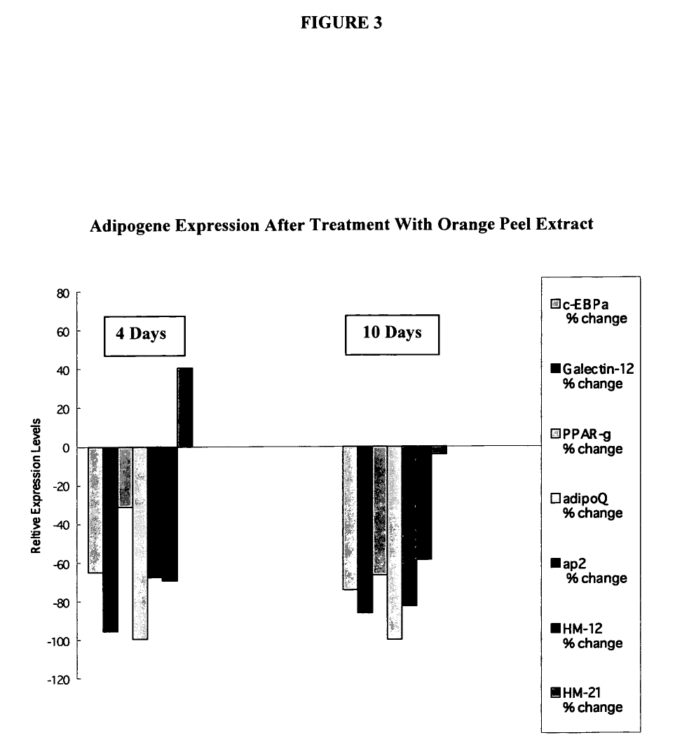

Results: FIG. 2 provides the changes in RNA levels observed in control cells treated with differentiation factor of day 0 as the cells differentiate into adipocytes by day 10. These cells were not treated with test extract. For cells treated with test (i.e., orange peel) extract, RNA levels were calculated as percent changes relative to the RNA levels observed in control cells for the corresponding day. For example, FIG. 3 provides the percent changes in RNA levels for test extract on days 4 and 10.

As indicated in FIG. 3, orange peel extract represses the expression of all but one of the adipogenes on day 4 and all the adipogenes on day 10. Visual microscopy showed that fat accumulation in these cells is very much reduced at all times in comparison to control cells.

Additional adipocyte gene expression/cell differentiation assays were performed on orange peel extract to confirm these results.

8.4. HMGA2 Assay

The high mobility group protein A2 (HMGA2), is a preadipocyte specific transcription factor induced in response to high-fat diets (Anand and Chada (2000) Nat. Genet. 24:377-380). Mice lacking the HMGA2 have 5-10% reduced fat compared to normal mice. Extracts that repress HMGA2 expression are likely to reduce adipose tissue formation.

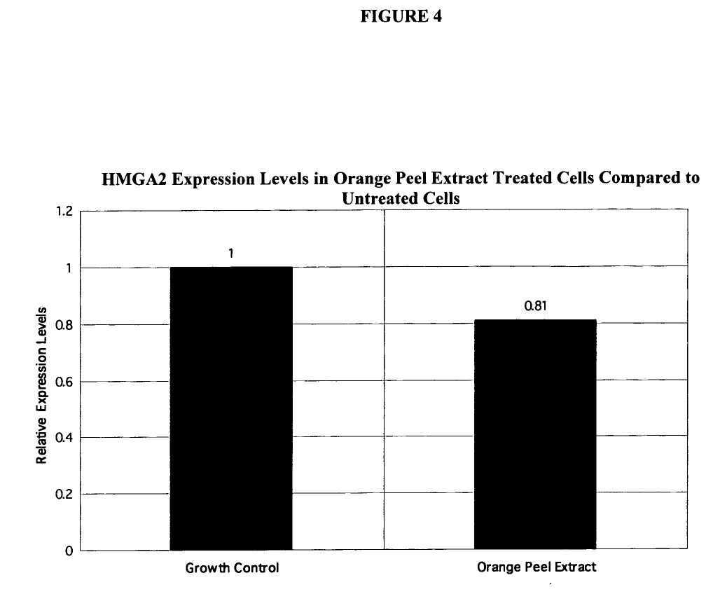

Murine 3T3-L1 cells were plated in 6 well plates with each well seeded with 200,000 cells and were grown in the presence of PMF-containing orange peel extract (20 μg/mL) or DMSO (control). After six hours of treatment, cells were collected and subjected to RNA extraction to determine HMGA2 expression using the procedures described for “gene expression detection” in Section 8.3 above. Data were normalized to control treated cells. As indicated in FIG. 4, orange peel extracts had some effect in reducing HMGA2 expression levels in treated cells as compared to control cells.

8.5. In Vitro Fat Accumulation Assay

This example demonstrates the effects of PMF-containing compositions on the inhibition of fat accumulation in adipocytes.

PMF-containing compositions comprising a 40% PMF fraction (“40% OPE”), a 70% PMF fraction (“70% OPE”), or comprising isolated compounds of 5,6,7,8,3′,4′-hexamethoxyflavone (nobiletin) or 5-hydroxy-6,7,8,3′,4′-pentamethoxyflavone (auranetin), were prepared from orange peel extracts as described above.

Adipocytes (3T3-L1 cells) were grown and subjected to standard differentiation-inducing agents, as described in Section 8.3 above, where adipocyte differentiation and accumulation of fat into the adipocytes was allowed to proceed in the absence or presence the presence of various concentrations of PMF-containing compositions. Visual microscopy of the cells stained with Oil Red O, which preferentially stains fat, was used to determine fat accumulation. Results are shown in Table 1, where observed staining intensity of accumulated fat is represented on a scale from “+++++” indicating maximal staining intensity to “+” indicating least staining intensity, and “n.d.” indicating treatments that were not determined.

TABLE 1
Fat Accumulation in Adipocytes

| | No | PMF-containing composition | | | | |
|---|---|---|---|---|---|---|
| | Treatment | 10 μg | 20 μg | 50 μg | 100 μg | 200 μg |
| 70% OPE | +++++ | +++++ | n.d. | +++ | + | + |
| 40% OPE | +++++ | +++++ | n.d. | +++ | +++ | + |
| nobiletin | +++++ | ++++ | ++++ | n.d. | n.d. | n.d. |
| auranetin | +++++ | ++++ | ++ | n.d. | n.d. | n.d. |

These results demonstrate that PMF-containing compositions are effective in inhibiting adipocyte development and/or fat accumulation in adipocytes.

8.6. In Vivo Studies

The following example demonstrates that introducing PMF-containing compositions into an animal diet can be effective in preventing weight gain and in promoting weight loss attributable to decreases in adipose tissue mass without loss of lean body mass. The male C57BL6/J mice used in this example have been shown to become obese, hyperglycemic, and hyperinsulinemic in a manner similar to humans and are a recognized model of human diet-induced obesity. See, e.g., Surwit et al. (1988) Diabetes 37: 1163-1167; Frederich et al. (1995) Nat. Med. 1:1311-1314; Parekh et al. (1998) Metabolism 47:1089-1896; El-Haschimi et al. (2000) J. Clin. Invest. 105:1827-1832; Lin et al. (2000) Int. J. Obes. Relat. Metab. Disord. 24:639-646.

The PMF-containing composition used in this example was the 70% OPE composition prepared as described above.

Mice and Diets: Male C57B1/6J mice were obtained from the Jackson Labs at three weeks of age. After one week of acclimation on a standard diet, the mice were randomly assigned into 4 groups: standard diet, standard diet+70% OPE, high fat diet, high fat diet+70% OPE. Standard diet mice were fed Research Diets D12450B (10 kcal % fat) (Van Heek et al. (1997) J. Clin. Invest. 99: 385-90). High fat diet mice were fed Research Diets D12451 (45 kcal % fat) (Van Heek et al. (1997) J. Clin. Invest. 99: 385-90). Diets containing 70% OPE were formulated by Research Diets and contained 2 g 70% OPE/1.055 kg standard diet and 2 g 70% OPE/858 g high fat diet, respectively, to match 70% OPE dosage to caloric density. Mice were housed three per cage in a temperature controlled room and were maintained in a 14/10-hour light dark cycle. Mice had ad libitum access to food and water. Mice were weighed weekly at the same time and food consumption/cage was determined at that time.

The mice gain weight in a biphasic pattern, with a larger body weight gain over the first 12 weeks followed by a decreased weight gain over subsequent weeks (Winzell and Ahren (2004) Diabetes 53 Suppl. 3:S215-S219). Therefore, these experiments were conducted during the first, high weight-gain phase in order to maximize the probability of observing a significant difference between the treated and untreated groups.

Lean Body Mass Analysis and % Body Fat: Lean body mass and percent fat was analyzed for 5 mice from each experimental group with the PIXImus Dual Energy X-ray Absorptiometry (DEXA) system (GE Healthcare, Waukesha, Wis.). The mice were analyzed following either 16 or 24 weeks of dietary treatment. For PIXImus analysis, 5 mice from each group were anaesthetized with an IP injection of 2.5 g % avertin (0.01 microliters/gram of body weight) and the analysis was performed as per the manufacturer's instructions. The system is based on the differential attenuation of low- and high-energy X-rays by the tissues in the scan area. Energy is attenuated in proportion to tissue density, and this information is used by the detector and associated software in conjunction with tissue calibration phantoms, to assess body composition. Fat mass consists primarily of adipose tissue. Lean mass consists primarily of skeletal muscle, blood and bone, but also organs, tendons, cartilage and body water.

Following the diet treatments, the animals were anaesthetized by isofluorane inhalation. The animals were sacrificed by cervical dislocation and the inguinal, epididymal, and retroperitoneal fat pads were dissected out and weighed. Samples of the mesenteric fat depot were also collected. Following weighing, RNA was extracted from the tissues as described above.

Carcass analysis on mice from the switched diet groups were performed using the Bruker mini-spec TD NMR analyzer (Bruker Optics, Billerica, Mass.) according to the manufacturer's instructions.

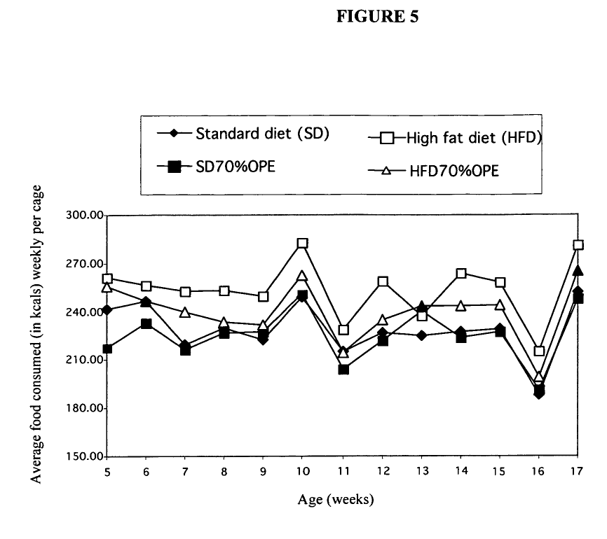

Results: Groups of 12-18 mice were studied for food consumption by placing three mice from a single group in a cage and measuring food consumed by the mice in the cage each week. Mice were place on a standard diet (“SD”), standard diet supplemented with 70% OPE (“SD70% OPE”), high fat diet (“HFD”) or high fat diet supplemented with 70% OPE (“HFD70% OPE”). Formulations of these diets are described above. The analysis of caloric consumption showed no significant difference in caloric intake between the SD and SD70% OPE groups (FIG. 5). Thus, 70% OPE did not in and of itself lead to hypophagia, either through unpalatability or through an adverse effect on the animals. While there was a trend toward higher caloric intake between the HFD and SD groups, it did not reach statistical significance. Thus, addition of 70% OPE to either the standard diet or the high fat diet did not result in any significant hypophagia.

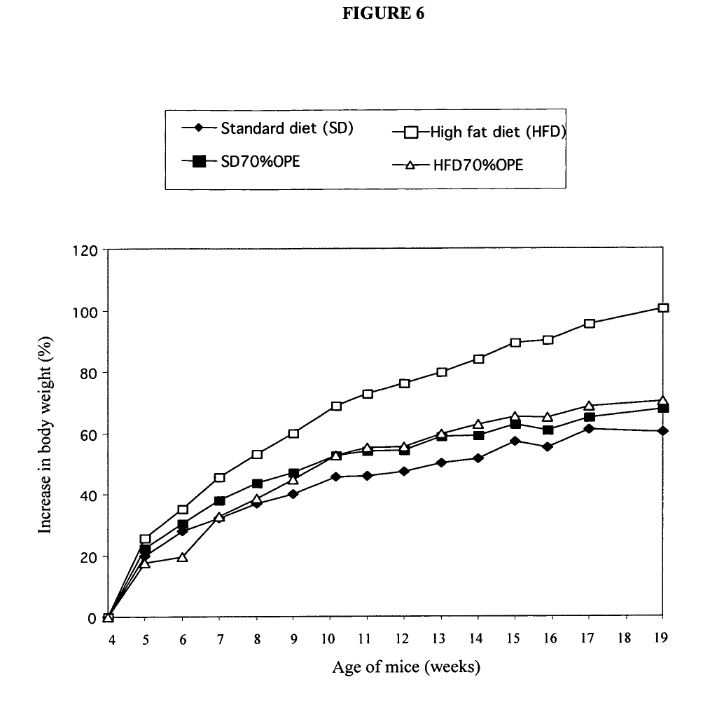

As shown in FIG. 6, significant differences in weight gain between the SD and HFD groups became apparent by week 7 of age (p<0.05) and continued throughout the course of the study. The HFD70% OPE mice showed a significant decrease in weight gain as compared to the HFD mice by week 5 of age (p<0.05) (FIG. 6). This difference was not due to a decrease in caloric intake (FIG. 5). These results show that a PMF-containing composition can prevent weight gain in a subject on a high fat diet.

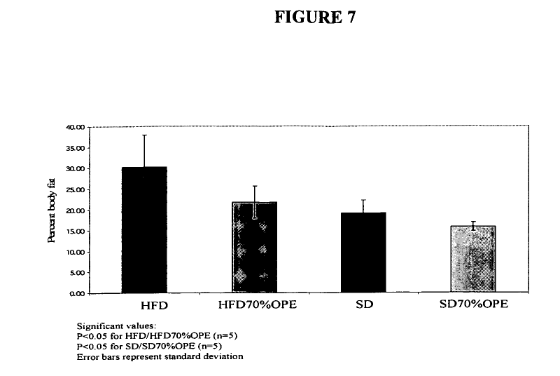

The difference in weight gain of the HFD70% OPE group as compared to the HFD group was investigated to determine if the reduced weight gains in the HFD70% OPE group were attributable to a decrease in fat mass versus a decrease in lean body mass. The fat mass of a cohort of animals (n=5 mice per group) was determined by dual X-ray absorptiometry (DEXA) using a PIXImus scanner. The percentages of body fat determined for each group are provided in FIG. 7. There was a significant decrease in percent body fat in the HFD70% OPE group as compared to the HFD group. There was no significant difference in percent body fat between the SD group and the HFD70% OPE group. Thus, addition of 70% OPE to the high fat diet prevented the increase in % body fat attributable to the high fat diet. These results indicate that a PMF-containing composition prevents weight gain attributable to a high fat diet largely by preventing fat accumulation.

A small but significant decrease in % body fat in the SD70% OPE animals as compared to the SD animals was observed (FIG. 7), though there was no significant difference in body weight (FIG. 6). These results are consistent with the possibility that a PMF-containing composition promotes the development of lean body mass in a subject.

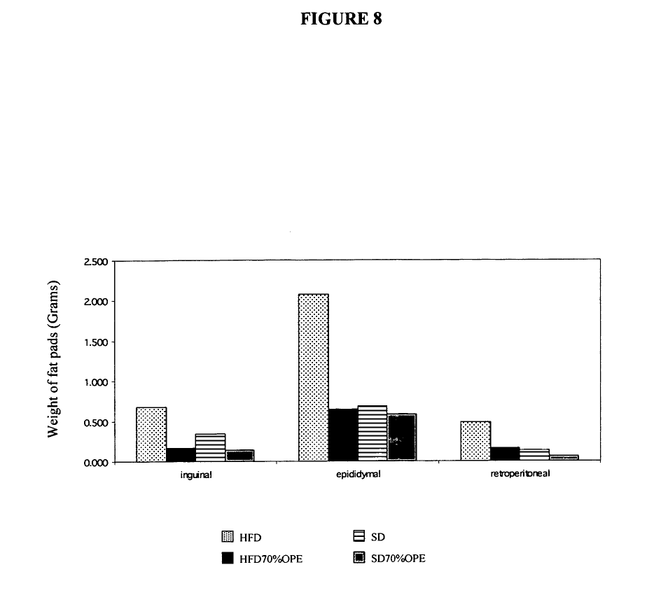

In order to assess the effects of 70% OPE on individual fat pads, two animals from each group were sacrificed and their inguinal, epididymal, and retroperitoneal fat pads were dissected out and weighed to assess any differences. The results are shown in FIG. 8. Though the cohort size was not large enough to provide any statistical analysis, the pattern was clearly for a large reduction in fat pad weight in the HFD70% OPE mice as compared to the HFD mice, with little difference among the HFDWGO201, SD, and SD70% OPE groups consistent with the body weight and PIXImus analyses.

Glycemic Parameters: It has been suggested that under certain circumstances subjects that are unable to expand their adipose mass will develop type II diabetes. See Danforth (2000) Nat. Genet. 26:13. The results provided below, however, indicate that subjects eating diets enriched with PMF-containing compositions do not appear to be more likely to develop diabetes.

To assess the likelihood that the mice used in the examples described herein would develop type II diabetes, gross examination of the livers of animals fed HFD70% OPE was performed, which did not reveal any increase in apparent steatosis as compared to HFD animals as would be expected in the case of overt lipodystrophy.

In addition, the glycemic status of the mice was determined by measurement of both fasting glucose using the Bayer Ascencia glucometer and measurement of glycated hemoglobin (HbA1c) using the Bayer DCA 2000+HbAlc analyzer (Bayer HealthCare, Tarrytown, N.Y.). All analyses were completed following a 4 hour fast, and all samples were collected via tail vein bleed.

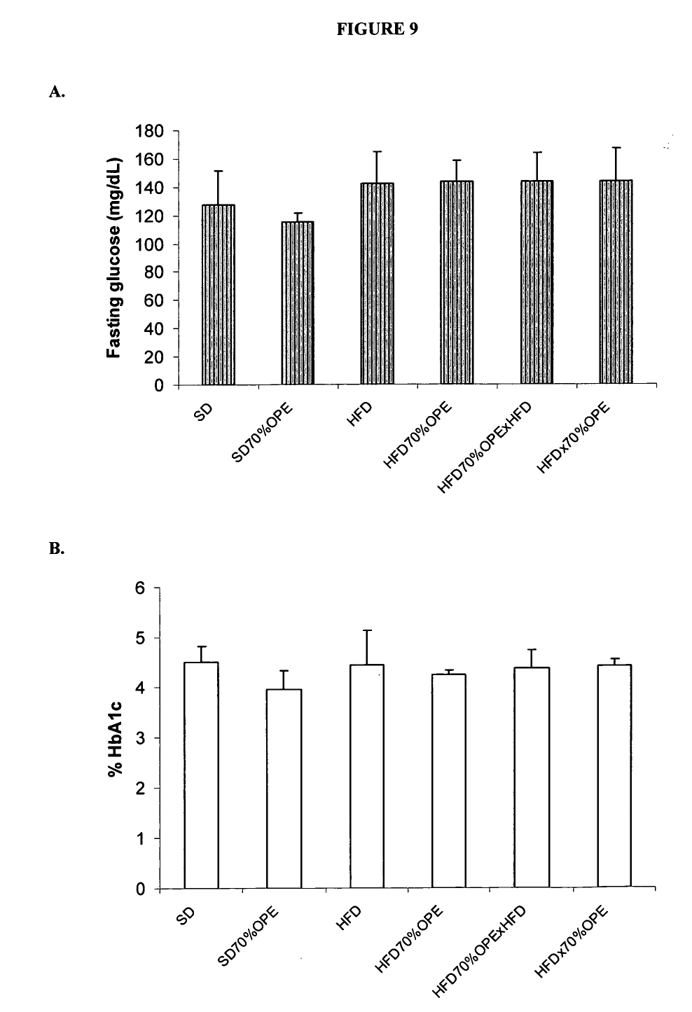

No significant difference in fasting glucose was observed between the SD group and the HFD group (FIG. 9A). There was a significant difference in fasting glucose between the SD70% OPE and HFD70% OPE groups. HbA1c glycation was measured since plasma glucose levels only represent a snapshot of the animals' current glucose status. The amount of glycated hemoglobin as a percentage of total hemoglobin correlates directly with average plasma glucose concentration over the previous two months. For HbA1c analysis, there was no significant difference amongst any of the groups with the exception of animals fed a standard diet plus 70% OPE that had a significant decrease in HbAIc (FIG. 9B). This suggests that, at least on a standard diet, 70% OPE may actually cause an improvement in glycemic control over the long term.

Diet Switching: At approximately 19 weeks of age, the impact of switching diets was assessed (Bush et al. (2006) Endocrine 29:375-382). Standard diet mice were switched to standard diet+70% OPE and vice versa. Likewise, high fat diet mice were switched to high fat diet+70% OPE and vice versa. In this example, mice were maintained on diet for 19 weeks and then fed a different diet for an additional 8 weeks. Food consumption and body weight measurements were taken as described above.

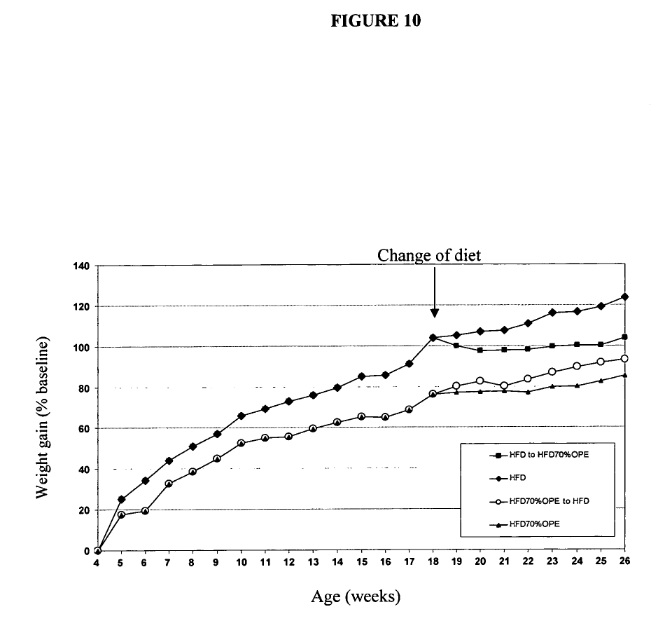

The SD and SD70% OPE groups did not differ significantly in calorie consumption or weight gain throughout the course of the initial 19 weeks, and switching the diets for the two groups had no apparent effect. However, significant changes were observed when the HFD mice were switched to HFD70% OPE and the HFD70% OPE mice were switched to HFD. Switching the HFD70% OPE mice to HFD had the effect of increasing the weight of the mice to statistically indistinguishable from the HFD mice (FIG. 10). By contrast, switching the HFD mice to HFD70% OPE (HFD to HFD70% OPE) resulted in a significant weight loss from HFD and from HFD70% OPE (FIG. 10). Thus, these data suggest that not only can 70% OPE prevent the weight gain observed upon the consumption of a high fat diet, but that it can reverse the weight gain in high fat diet fed animals even while they continue to consume a high fat diet.

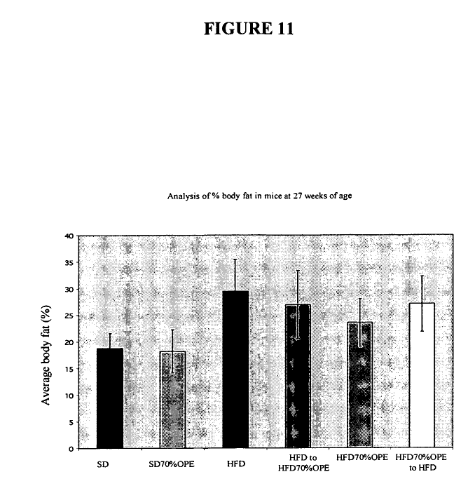

Following the procedures described above for using the PIXImus Dual Energy X-ray Absorptiometry (DEXA) system to assess lean body mass and percent body fat, an analysis was done on the animals to determine if the changes in weight correlated with changes in percent fat mass. The HFD70% OPE mice switched to HFD showed an increase in percent body fat, while the HFD animals switched to HFD70% OPE showed a moderate decrease in percent body fat (FIG. 11).

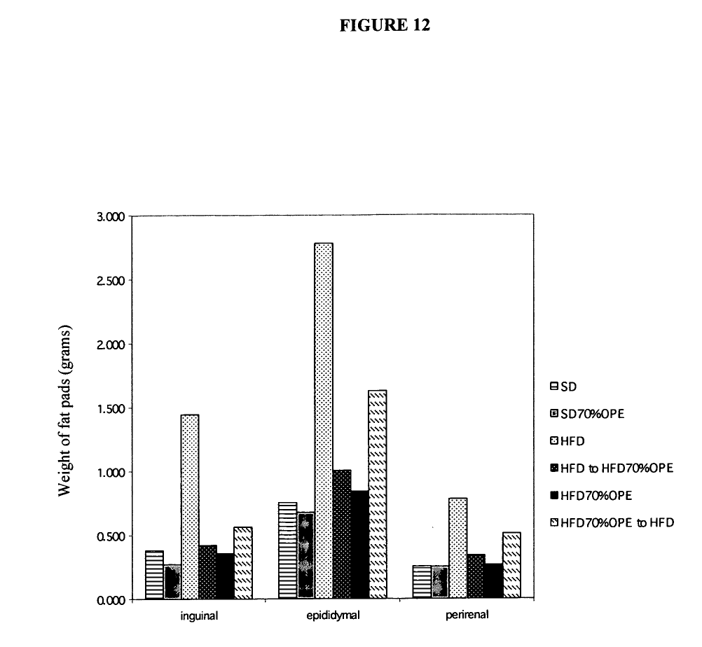

Analysis of individual fat pads from the animals confirmed the results seen in the PIXImus analysis, with decreases in all three fat pads seen in the HFD to HFD70% OPE group and increases in all three fat pads in the HFD70% OPE to HFD group (FIG. 12).

Taken together, the data indicate that including PMF-enriched compositons in the context of a high fat diet results in a significant decrease in weight gain. Moreover, in diet-induced obese, adult animals the administration of a PMF-containing composition caused a significant decrease in weight. Both the prevention of weight gain and promotion of weight loss could be attributed to a decrease in adipose tissue mass with no loss of lean body mass. Importantly, the decrease in adiposity was not accompanied by any apparent signs of diabetes or loss of glycemic control. It was also noted that mice switched from a PMF-enriched high fat diet to a non-PMF-enriched high fat diet gained weight similar to their counterparts on a non-PMF-enriched high fat diet indicating that the effects of PMF-containing composition on weight prevention or loss can be reversed with the removal of the PMF-containing composition from the diet.

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed:

1. A method of managing fat accumulation in a subject comprising administering to the subject an effect amount of a composition comprising an orange peel extract having a polymethoxylated flavone (PMF) fraction of at least about 30% (w/w) (dry weight) to about 75% (w/w) (dry weight) thereby managing fat accumulation in the subject.

2. The method of claim 1, wherein the composition comprises a PMF fraction of at least about 40% (w/w) (dry weight) to about 75% (w/w) (dry weight).

3. A method of managing fat accumulation in a subject comprising administering to the subject an effect amount of a composition comprising a polymethoxylated flavone (PMF) fraction and a non-PMF fraction, wherein the PMF fraction comprises at least one of
5,6,7,3′,4′-pentamethoxyflavone (sinensetin);
5,6,7,8,3′,4′-hexamethoxyflavone (nobiletin);
5,6,7,8,4′-pentamethoxyflavone (tangeretin);
5-hydroxy-6,7,8,3′,4′-pentamethoxyflavone (auranetin);
5-hydroxy-7,8,3′,4′-tetramethoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; or
7-hydroxy-3,5,6,3′,4′-pentamethoxyflavone,
thereby managing fat accumulation in the subject.

4. The method of claim 3, wherein fat accumulation in one or more adipocytes in the subject is managed.

5. The method of claim 4, wherein adipogenesis is reduced.

6. The method of claim 4, wherein fat accumulation in a mature adipocyte is reduced.

7. The method of claim 3, wherein the composition comprises a PMF fraction of about 40% (w/w) (dry weight) to about 75% (w/w) (dry weight).

8. The method of claim 7, wherein the composition of claim 1 is orally administered to the subject.

9. The method of claim 8, wherein the composition is in a dry form.

10. The method of claim 8, wherein the composition is in a liquid form.

11. The method of claim 8, wherein the subject is human.

12. The method of claim 11, wherein about 50 mg to about 2000 mg of the compound is administered to the subject per day.

13. The method of claim 12, wherein the composition is administered once a day for three to about 30 days.

14. The method of claim 2, wherein the polymethoxylated flavone (PMF) fraction comprises at least two PMFs selected from the group consisting of
5,6,7,3′,4′-pentamethoxyflavone (sinensetin);
5,6,7,8,3′,4′-hexamethoxyflavone (nobiletin);
5,6,7,8,4′-pentamethoxyflavone (tangeretin);
5-hydroxy-6,7,8,3′,4′-pentamethoxyflavone (auranetin);
5-hydroxy-7,8,3′,4′-tetramethoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; and
7-hydroxy-3,5,6,3′,4′-pentamethoxyflavone.

15. The method of claim 2, wherein the PMF fraction comprises
5,6,7,3′,4′-pentamethoxyflavone (sinensetin);
5,6,7,8,3′,4′-hexamethoxyflavone (nobiletin);
5,6,7,8,4′-pentamethoxyflavone (tangeretin);
5-hydroxy-6,7,8,3′,4′-pentamethoxyflavone (auranetin);
5-hydroxy-7,8,3′,4′-tetramethoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; and
7-hydroxy-3,5,6,3′,4′-pentamethoxyflavone.

16. The method of claim 2, wherein the PMF fraction consists of
5,6,7,3′,4′-pentamethoxyflavone (sinensetin);
5,6,7,8,3′,4′-hexamethoxyflavone (nobiletin);
5,6,7,8,4′-pentamethoxyflavone (tangeretin);
5-hydroxy-6,7,8,3′,4′-pentamethoxyflavone (auranetin);
5-hydroxy-7,8,3′,4′-tetramethoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; and
7-hydroxy-3,5,6,3′,4′-pentamethoxyflavone.

17. A method of managing rebound weight gain in a subject comprising administering to the subject in need thereof an effect amount of a composition comprising a PMF fraction wherein the PMF fraction comprises at least one of
5,6,7,3′,4′-pentamethoxyflavone (sinensetin);
5,6,7,8,3′,4′-hexamethoxyflavone (nobiletin);
5,6,7,8,4′-pentamethoxyflavone (tangeretin);
5-hydroxy-6,7,8,3′,4′-pentamethoxyflavone (auranetin);
5-hydroxy-7,8,3′,4′-tetramethoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; or
7-hydroxy-3,5,6,3′,4′-pentamethoxyflavone,
thereby managing rebound weight gain in the subject.

18. The method of claim 17, wherein the subject is a human who has lost weight from a restricted calorie diet.

19. The method of claim 17, wherein the composition comprises a PMF fraction of about 30% (w/w) (dry weight) to about 75% (w/w) (dry weight).

20. The method of claim 17, wherein the PMF fraction comprises at least two PMFs selected from the group consisting of
5,6,7,3′,4′-pentamethoxyflavone (sinensetin);
5,6,7,8,3′,4′-hexamethoxyflavone (nobiletin);
5,6,7,8,4′-pentamethoxyflavone (tangeretin);
5-hydroxy-6,7,8,3′,4′-pentamethoxyflavone (auranetin);
5-hydroxy-7,8,3′,4′-tetramethoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; and
7-hydroxy-3,5,6,3′,4′-pentamethoxyflavone.

21. The method of claim 17, wherein the PMF fraction comprises
5,6,7,3′,4′-pentamethoxyflavone (sinensetin);
5,6,7,8,3′,4′-hexamethoxyflavone (nobiletin);
5,6,7,8,4′-pentamethoxyflavone (tangeretin);
5-hydroxy-6,7,8,3′,4′-pentamethoxyflavone (auranetin);
5-hydroxy-7,8,3′,4′-tetramethoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; and
7-hydroxy-3,5,6,3′,4′-pentamethoxyflavone.

22. The method of claim 2, wherein the PMF fraction consists of
5,6,7,3′,4′-pentamethoxyflavone (sinensetin);
5,6,7,8,3′,4′-hexamethoxyflavone (nobiletin);
5,6,7,8,4′-pentamethoxyflavone (tangeretin);
5-hydroxy-6,7,8,3′,4′-pentamethoxyflavone (auranetin);
5-hydroxy-7,8,3′,4′-tetramethoxyflavone;
5,7-dihydroxy-6,8,3′,4′-tetramethoxyflavone;
5,7,8,3′,4′-pentamethoxyflavone;
5,7,8,4′-tetramethoxyflavone;
3,5,6,7,8,3′,4′-heptamethoxyflavone;
5-hydroxy-3,6,7,8,3′,4′-hexamethoxyflavone;
5-hydroxy-6,7,8,4′-tetramethoxyflavone;
5,6,7,4′-tetramethoxyflavone;
7-hydroxy-3,5,6,8,3′,4′-hexamethoxyflavone; and
7-hydroxy-3,5,6,3′,4′-pentamethoxyflavone.

23. A method of losing weight, managing weight or preventing weight gain in a subject comprising administering to the subject an effective amount of a composition comprising a polymethoxylated flavone (PMF) fraction of at least about 30% (w/w) (dry weight) to about 75% (w/w) (dry weight).

24. The method of claim 23, wherein the composition is administered in daily dose over a period of time of about one week to about eight weeks.

25. A method of increasing lean body or muscle mass in a subject comprising administering to the subject an amount of a composition effective to reduce fat accumulation in the subject wherein the composition comprises a polymethoxylated flavone (PMF) fraction of at least about 30% (w/w) (dry weight) to about 75% (w/w) (dry weight), thereby increasing lean body or muscle mass in the subject.

26. A method of reducing waist circumference in a subject comprising administering to the subject an effective amount of a composition comprising a polymethoxylated flavone (PMF) fraction of at least about 30% (w/w) (dry weight) to about 75% (w/w) (dry weight).

27. A method of increasing metabolic rate in a subject comprising administering to the subject an effective amount of a composition comprising a polymethoxylated flavone (PMF) fraction of at least about 30% (w/w) (dry weight) to about 75% (w/w) (dry weight).

28. A method of preventing fat accumulation in an adipocyte comprising contacting the adipocyte with a composition comprising a polymethoxylated flavone (PMF) fraction.

29. The method of claim 28, wherein the contacting occurs in vitro.
